(19) United States
(12) Patent Application Publication
(10) Pub. No.: US 2003/0118628 A1
(43) Pub. Date: Jun. 26, 2003

(54) Confectionery product having a salivation region and an oral comfort region

(76) Inventors: Nurhan Pinar Tutuncu, Flanders, NJ (US); Sonia Hartunian-Sowa, Morristown, NJ (US); Julie A. Bratton, Ledgewood, NJ (US); Kevin J. Stanton, Califon, NJ (US); Jean Lee, Bridgewater, NJ (US)

Correspondence Address: FITZPATRICK CELLA HARPER & SCINTO, 30 ROCKEFELLER PLAZA, NEW YORK, NY, 10112, US

(21) Appl. No.: 09/825,992

(22) Filed: Apr. 5, 2001

(51) Int. Cl. A61K 9/00; A23G 3/00

(52) U.S. Cl. 424/440; 426/660

(57) ABSTRACT

A confectionery product for relief of drymouth is disclosed having a salivation region and a separate and distinct oral comfort region. Also disclosed are methods of making and using the confections of the invention.

### BACKGROUND OF THE INVENTION

1. Field of the Invention

This invention is directed to confections which may be used to alleviate dry mouth (xerostomia). In particular, the present invention is directed to confections having both a salivation agent to promote salivation and an oral comfort ingredient to make the mouth of a user feel more comfortable. Significantly, the salivation agent is located in a separate and distinct region of the confection from the oral comfort ingredient. The present invention is also directed to methods for making and using these confections.

2. Related Background Art

Dry mouth is a common problem throughout the world which is caused by a variety of environmental, emotional and physiological factors. This condition, also known as xerostomia, exhibits physical symptoms such as decreased amount of saliva, decreased mouth moistness, inability to move the tongue freely, speech problems, incomplete digestion of food, swallowing problems, increased breath odor, increased risk of oral health problems and general throat irritation.

Depending on the severity of the dry mouth symptoms, there are currently a variety of products that can be used to alleviate this condition. For example, the prescription drug pilocarpine and general saliva substitutes (liquids and sprays) can be used by individuals exhibiting severe, clinical cases of dry mouth. In addition, apparatus exist that can be inserted into the mouth to help introduce artificial saliva or compounds that stimulate salivation.

Consumers with less severe cases of dry mouth have reported using hard candies, cough drops, gums, mints and lozenges to alleviate their condition. Although these products can offer some relief from dry mouth discomfort, a product having superior product characteristics, e.g., convenience of use and excellent taste, in a single product would be highly desirable.

Various gum compositions have been described for stimulating salivation. For example, U.S. Pat. No. 5,571,528 to Lee describes a pilocarpine containing chewing gum for stimulating salivation. The pilocarpine that reaches the salivary glands through the oral mucosa stimulates salivation.

U.S. Pat. No. 4,088,788 to Ream describes the use of a chewing gum composition containing an organic acid in combination with saccharin to stimulate salivation beyond that attributable to the act of chewing or the effects of the individual ingredients. The gum in this invention is intended for use by athletes who have dry mouth after exercising. A sweetener is added to provide a quick source of energy, and salts may be added to replace potassium and sodium ions lost in perspiration during exercise.

U.S. Pat. No. 4,997,654 to Corsello describes a method for treating xerostomia with a chewing gum or candy containing xylitol, a 5-carbon sugar alcohol, as a bulk filler and sweetener to assist salivation.

International application WO89/09594 to Hoerman describes a method for treating xerostomia (not caused by exercising) through the use of a gum containing a relatively insoluble hydrophobic salivation agent. The salivation agent is retained in the chewing gum base and released over an extended period of time during chewing. The gum base should be free of calcium carbonate or other alkaline fillers which tend to neutralize the salivation agent. Calcium and phosphorus may also be added to prevent dissolution of tooth enamel.

Lozenges for stimulating saliva have also been described. For example, U.S. Pat. No. 5,156,845 to Grodberg describes a lozenge containing a base, a sugarless sweetener, an salivation agent, and fluoride to inhibit tooth decay. The base may be composed of a pharmaceutically acceptable sugarless chewing gum.

U.S. Pat. No. 5,614,207 to Shah describes a dry mouth lozenge for stimulating the flow of saliva. The lozenge is made of a lozenge base, a demulcent, a humectant, and an salivation agent. Upon hydration by saliva, the demulcent imparts a wet, slippery mouth feel. A humectant is employed to further enhance the moisturizing properties of the lozenge.

International application WO81/02977 to Pedersen discloses a sterile, preserved mucine-containing solution for application to mucous membranes. Individuals suffering from xerostomia can use this solution as artificial saliva.

It would be desirable to produce a confection to alleviate dry mouth that exhibits superior product characteristics such as a combination of ease of use with acceptable taste. It would be further desirable for the confection to have both a salivation agent to promote salivation and an oral comfort ingredient to make the mouth more comfortable. However, in the case were the salivation agent is an acidulent, its combination with an acid sensitive oral comfort ingredient can be deleterious. Previous attempts to overcome this problem associated with combining certain salivation agents with acid-sensitive ingredients have focused on the use of co-processed compositions whereby the salivation agent is combined with a water-soluble crystalline compound.

International application WO99/59427 to Le discloses co-processed compositions containing at least one salivation agent and a water-soluble crystalline compound for use in confectionery, dentifrice, or pharmaceutical products containing acid-sensitive additives, such as flavors. These co-processed compositions are made by combining the salivation agent with the water-soluble crystalline compound to form a mixture, which is then formed into granules or agglomerates and added to the remaining product. These co-processed composition suffer from moisture sensitivity because they must be prepared with components that are low in moisture and maintained in a low moisture environment for the salivation agent to remain separate from the acid-sensitive additives. It would be desirable to produce a confection without the need to resort to these co-processed crystalline compositions.

While there are many known products to treat xerostomia, a confectionery product for treating dry mouth that combines in a single package, acceptable taste, convenience and portability with increased salivation and enhanced oral comfort would be highly desirable.

### SUMMARY OF THE INVENTION

This invention is directed to a confectionery product comprising: (i) a salivation region comprising a salivation agent in an amount effective to aid in the stimulation of the flow of saliva in an oral cavity and a salivation region confectionery base; and (ii) an oral comfort region that is separate and distinct from said salivation region, said oral comfort region comprising an oral comfort ingredient in an amount effective to comfort said oral cavity and an oral comfort region confectionery base. The separation of the salivation agent and the oral comfort ingredient minimizes the potential deleterious problems that can result when the oral comfort ingredient is acid sensitive.

More significantly, it has been surprisingly discovered that having the salivation agent concentrated in one region of the product enhances the initial salivation effect and promotes mechanical action or movement of the piece throughout the mouth. In particular, because of the separation of the two regions, greater amounts of salivation agent may be used resulting in an increased initial impact causing enhanced oral manipulation, but still with acceptable taste.

In a particularly preferred embodiment of the present invention, a cooling compound is included in the salivation region. It is believed that the presence of the cooling compound enhances the effects of salivation.

The present invention is also directed to a method for preparing confectionery products having a separate salivation region and an oral comfort region. The method of the invention comprises the steps of: (i) mixing a salivation agent with a salivation region confectionery base to form an salivation agent containing confectionery base; (ii) mixing an oral comfort ingredient with an oral comfort region confectionery base to form an oral comfort ingredient containing confectionery base; and (iii) forming said confectionery product with said salivation agent containing confectionery base and said oral comfort ingredient containing confectionery base in a manner that maintains said salivation agent containing confectionery base and said oral comfort ingredient containing confectionery base in separate and distinct regions of said confectionery product. The resulting confectionery product has the salivation agent concentrated in one region of the product while the oral comfort ingredient is concentrated in a separate region of the product.

A preferred embodiment for the method of making the confectionery product utilizes a mold with a ridge running along its bottom inner surface which bisects the mold and assists in preventing the oral comfort ingredient from mixing with the salivation agent.

The present invention is also directed to a method for treating dry mouth (xerostomia) which comprises introducing into the oral cavity a confectionery product containing a salivation agent concentrated in one region of the confectionery product and an oral comfort ingredient concentrated in a separate region of the product. In a particularly preferred embodiment, a cooling compound is added to the salivation region to enhance salivation.

### DETAILED DESCRIPTION OF THE INVENTION

The present invention is a confection that promotes salivation while comforting, e.g., lubricating and/or moisturizing, the mouth that is superior to traditional hard candies, cough drops, gums, mints and lozenges. The invention combines a salivation agent with an oral comfort ingredient in a confectionery base with the salivation agent concentrated in an area of the product that is distinct from the area containing the oral comfort ingredient. The confectionery product of this invention may be used by individuals suffering from dry mouth (xerostomia).

The confectionery products of this invention have a confectionery base in both the salivation region and the oral comfort region. The confectionery base in each region of the product may be the same or different, but preferably is the same. The confectionery base may be a carbohydrate or carbohydrate derivative such as sugar base, a sugarless base or a gum. Preferably the confectionery base is a sugar base or a sugarless base.

Exemplary sugar bases include a sugar selected from the group consisting of sucrose, glucose, fructose, maltose, corn syrup and mixtures thereof. Preferably, the sugar used as the confectionery base in both the salivation region and the oral comfort region is a mixture of sucrose and corn syrup.

In yet another embodiment, the confectionery base is a sugarless base. Exemplary sugarless bases may be selected from the group consisting of isomalt, erythritol, hydrogenated starch hydrolysates, sorbitol, xylitol, mannitol and mixtures thereof. A preferred sugarless base is isomalt. Isomalt is a sugar alcohol made from sucrose. It is non-cariogenic, does not lead to appreciable increases in blood sugar or insulin levels, and has a caloric utilization of 50%. Small amounts of artificial sweeteners such as acesulfame K (AceK), sucralose, saccharin, cyclamate and aspartame may be added to the sugarless base to enhance its sweetness.

The confectionery base of this invention is generally present in an amount of about 50% to about 99.5% by weight of the total composition. More preferably the confectionery base is present in an amount of about 80% to about 98%, most preferably about 90% to about 97% by weight of the total composition.

The salivation region of the confectionery product will contain an salivation agent in combination with the confectionery base. The salivation agent is present in an amount effective to promote salivation in the oral cavity. Any orally acceptable compound that promotes salivation in the oral cavity may be used as a salivation agent. Exemplary salivation agents include acidulents, cooling compounds, salts, salt enhancers, monosodium glutamate (MSG), MSG enhancers, flavors and mixtures thereof. Acidulents are a preferred salivation agent. Exemplary acidulents include citric acid, malic acid, succinic acid, adipic acid, tartaric acid, acetic acid, lactic acid and mixtures thereof.

The salivation agent is generally present in an amount ranging from about 0.01% to about 4% by weight of the salivation region, and more preferably in an amount of about 1.5 to about 2% by weight of the salivation region. The concentration of the salivation agent in the salivation region may vary depending on the weight ratio of the salivation region(s) to the oral comfort region(s) of the product.

In a preferred embodiment the weight ratio of the salivation region to the oral comfort region will be about 50:50. It should be clear that the present invention may include multiple salivation and oral comfort regions in a single confectionery product. In addition, the confectionery product may include regions that are neither salivation regions or oral comfort regions so long as there is at least one salivation region and one oral comfort region which are separate and distinct regions from each other. The separation of these two regions is of particular significance in the present invention. Preferably, each such region has a surface on the exterior of the confectionery product. In a preferred embodiment of this invention the confectionery product is comprised solely of a single salivation agent region that borders, but is separate from, a single oral region.

In a particularly preferred embodiment of the invention the salivation region includes a cooling compound, such as L-menthol, N-ethyl-p-methane-3-carboxamide, N,2,3-trimethyl-2-isopropyl butanamide and monomethyl succinate. See e.g., Parrish, M. A., “Market Warms To Physiological Coolants”, Manufacturing Chemist, pp. 31-32 (February 1987). Other exemplary cooling compounds for use in the present invention include “COOLER II” available from International Flavors and Fragrances, Inc. (IF&F), Dayton, N.J. and “PHYSCOOL” available from MANE USA Milford, Ohio. It is believed that a synergistic effect is provided by a mixture of an acidulent and the cooling compound, resulting in enhanced salivation.

Preferably, the cooling compound is present in an amount ranging from about 0 to about 0.2% by weight of the product. It is further preferred that the cooling compound be from about 0.01 to about 0.15% by weight of the product.

The oral comfort region of the confectionery product will contain an oral comfort ingredient in combination with an oral comfort region confectionery base. The oral comfort ingredient will be present in an amount effective to comfort, e.g., lubricate, coat and/or moisten, the oral cavity. The oral comfort region may also contain relatively small amounts of acidulents, e.g., in amounts effective to potentiate the flavor components. If desired, the oral comfort region may also contain a cooling compound.

The oral comfort ingredient may be selected from the group consisting of lipids, proteins, surfactants or mixtures thereof. Preferably the oral comfort ingredient is a lipid. The lipid useful in the present invention may be selected from the group consisting of partially hydrogenated palm kernel oil, medium chain triglycerides, coconut oil, anhydrous milk fat, cocoa butter, corn oil, palm oil, soybean oil, sunflower oil, canola oil and mixtures thereof. A particularly preferred lipid is partially hydrogenated palm kernel oil.

Generally, if a lipid is used as an oral comfort ingredient it will be present in an amount of about 1 to about 20%, preferably about 2 to about 5%, and more preferably about 3 to about 4% by weight of the confectionery product.

In yet another embodiment of the invention the oral comfort ingredient may be protein. Proteins may provide comfort to the oral cavity by moisturizing and/or forming a film that can protect and retain moisture. Exemplary proteins include casein, whey, mucins, egg, blood proteins and proteins processed by microparticulation. If a protein is used as an oral comfort ingredient it generally will be present in an amount ranging from about 0.25 to about 2.5% by weight of the confectionery product.

In a particularly preferred embodiment of this invention the oral comfort region will include a mixture of a lipid and a surfactant. The surfactant is selected from the group consisting of nonionic surfactants, anionic surfactants and amphoteric surfactants. Preferably the surfactant is a nonionic surfactant that is a monoglyceride or diglyceride fatty acid ester. Generally, if the surfactant is used then it is present in an amount of about 0.1 to about 4% by weight of the oral comfort region, preferably in an amount of about 0.3 to about 2% by weight of the oral comfort region, and most preferably about 0.5 to about 1% by weight of the oral comfort region. With respect to the entire confectionery product, the surfactant, if used, will generally be present in an amount of about 0.005 to about 2% by weight, preferably about 0.15 to about 1% by weight, and most preferably about 0.25 to about 0.5% by weight of the confectionery product.

In addition, other additives, such as colors, sweeteners and flavor components, may be added to the product to impart desirable taste and appearance. Such additives may be independently added to the salivation region and the oral comfort region of the confectionery product as desired. For example it may be desirable to add a colorant to one region while leaving a separate region colorless or clear. It may also be desirable to employ the same or different flavor components in both the salivation region and the oral comfort region.

This invention is also directed to a method of preparing a confectionery product having a salivation region and an oral comfort region that is separate and distinct from said salivation region. The method comprises the steps of forming an salivation agent containing confectionery base by mixing a salivation region confectionery base and a salivation agent and forming an oral comfort ingredient containing confectionery base by mixing an oral comfort ingredient with an oral comfort confectionery base. The confectionery of this invention is then formed in a manner that maintains the salivation agent containing confectionery base and the oral comfort ingredient containing confectionery base in separate and distinct regions of the confectionery product.

The salivation region confectionery base and the oral comfort region confectionery base may be comprised of the same or different confectionery base. Preferably, the confectionery base used in both regions is the same.

In a particularly preferred embodiment of the method of the invention the confectionery base is cooked and evaporated to a desired moisture content, e.g., about 0.5% to about 4% by weight. Once the confectionery base is in a molten state, the oral comfort ingredient as well as optional flavors and colors, are added to one portion of the cooked confectionery base (the “oral comfort ingredient containing confectionery base”), while the salivation agent as well as optional cooling compound, and other flavors, are added to a separate portion of the cooked confectionery base (the “salivation agent containing confectionery base”). As previously noted, if desired an optional cooling compound may be added to the oral comfort ingredient containing confectionery base.

Various techniques can be used in the method of this invention to form a confectionery in which the oral comfort ingredient containing confectionery base and salivation agent containing confectionery base are maintained as discrete and separate regions in the confectionery product. Such techniques include the use of molding and panning techniques, as well as other known confectionery manufacturing techniques. Significantly, the technique used must result in a confectionery product having at least two separate regions, one region containing a salivation agent and the other containing the oral comfort ingredient.

In a particularly preferred embodiment, the salivation agent containing confectionery base is co-deposited alongside the oral comfort ingredient containing confectionery base into separate regions of a rigid mold. The ingredients are then cooled and ejected from the mold to form the final product. The product can also be prepared by panning an inner core of a salivation or oral comfort region and an outer layer of the other region. Other types of hard candy confectionery products, e.g., pulled hard candy or formed hard candy, are also contemplated. Besides hard candy, the confectionery products of this invention may also include other candies such as gummy or toffee candies and gum, although hard candies are preferred. The preferred confectionery product of this invention has a concentrated region of a salivation agent adjacent to, but separate from, the concentrated region of oral comfort ingredient. Such a construct will minimize any deleterious effect that the salivation agent might have on the oral comfort ingredient. More significantly, the concentrated region of the product containing the salivation agent enhances the initial salivation effect and promotes mechanical action or movement of the piece throughout the mouth. The superior characteristics of the inventive product result in part because high concentrations of salivation agent may be used in the salivation region resulting in enhanced oral manipulation of the product, while producing acceptable product taste.

It should be apparent that more than two deposits can be made into a single mold cavity if desired. For example, a first deposit of the oral comfort ingredient containing confectionery base, a second deposit of the salivation agent containing confectionery base and a third deposit of a confectionery base containing neither an oral comfort ingredient or a salivation agent can be made into a single mold cavity. These deposits are made substantially simultaneously so that an integral confectionery product is formed. Of course it is also possible to make more than one deposit of the oral comfort ingredient containing confectionery base and/or more than one deposit of the salivation agent containing confectionery base in a single confectionery mold so long as at least two separate and distinct regions are formed. Preferably, however, the confectionery product has two separate regions, i.e., a salivation region and an oral comfort region that border each other.

In a particularly preferred embodiment, the molds used to form the confectionery product have a raised ridge along the interior bottom surface of the mold which helps separate the salivation agent from the oral comfort ingredient. The ridge helps guide the flow of molten syrup into the mold and maintain the separation of the ingredients while cooling. The ridge may be a straight line or have curvature.

The confectionery product of this invention may take any desired shape. Preferably a shape is chosen, e.g. an oval or elliptical shape, that provides mouth comfort while encouraging manipulation of the confectionery product in the mouth.

The confectionery products of this invention may be used to treat xertostomia, if desired. The Examples which follow are intended as an illustration of certain preferred embodiments and no limitation is implied.

### EXAMPLE 1

A confectionery having a sugar base was prepared as follows using the ingredients set forth in Table 1. First a raw syrup was prepared by blending the sugar, corn syrup and water to create an 80% solids syrup. The syrup was cooked to about 150° C. to reduce the water content to about 2%. The cooked syrup (confectionery base) was cooled to about 140° C. and divided into two approximately equal batches. One batch was used to prepare the oral comfort region and the other batch was used to prepare the salivation region.

The salivation agent containing confectionery base was prepared by first mixing the succinic acid with water to 85% solids. The acid water mixture was heated to 90° C. in order to dissolve the acid into solution. The acid solution, mint flavor and “COOLER II” cooling compound were then blended with one batch of the 140° C. cooked syrup.

The oral comfort ingredient containing confectionery base was prepared by blending the partially hydrogenated palm kernel oil with the nonionic surfactant after heating the fat and surfactant to about 40° C. Next the fat/surfactant blend, colorant and mint flavor were blended with the other batch of 140° C. cooked syrup.

The salivation agent containing confectionery base and the oral comfort ingredient containing confectionery base were poured into separate depository funnels and dual deposited side by side to a rigid mold with a ridge running along the center of the bottom inner surface of the mold to assist in placement of both the salivation agent containing confectionery base and the oral comfort ingredient containing confectionery base. The depositing temperature was about 125° C. to 135° C. The confectionery product was cooled to about 20° C. to 35° C. in about 15 minutes. The cooled confectionery product was then demolded.

TABLE 1
Sugar Candy—Mint Flavor

| | Ingredient (%) | Ingredient (%) |
|---|---|---|
| | one side | total |
| Cooked syrup | | |
| Sugar - 53.9% | | |
| Corn syrup solids - 44.1% | | |
| Water - 2.0% | | |
| Oral Comfort Region | | |
| Cooked syrup | 93.24 | 46.62 |
| Lipid¹ | 5.59 | 2.8 |
| Surfactant² | 0.70 | 0.35 |
| Colorant | 0.10 | 0.05 |
| Flavorant | 0.37 | 0.185 |
| Total | 100.000 | 50.000 |
| Salivation Region | | |
| Cooked syrup | 99.25 | 49.63 |
| Succinic Acid | 0.200 | 0.10 |
| “COOLER II”³ | 0.15 | 0.075 |
| Flavorant | 0.40 | 0.20 |
| Total | 100.000 | 50.000 |

¹Partially hydrogenated palm kernel oil
²Mono and diglyceride fatty acid ester sourced from Canola oil (available as “Myverol” 18-99 from Quest, Hoffman Estates, IL.
³A cooling compound available from IFF (Dayton, New Jersey).

### EXAMPLE 2

A confectionery having a sugar base was prepared in a manner substantially similar to Example 1 using the ingredients set forth in Table 2, with the exception that four acids were mixed with water and citrus flavor was added to the cooled syrup.

TABLE 2
Sugar Candy—Citrus Flavor

| | Ingredient (%) | Ingredient (%) |
|---|---|---|
| | one side | total |
| Cooked syrup | | |
| Sugar - 53.9% | | |
| Corn syrup solids - 44.1% | | |
| Water - 2.0% | | |
| Oral Comfort Region | | |
| Cooked syrup | 93.19 | 46.60 |
| Lipid¹ | 5.59 | 2.8 |
| Surfactant² | 0.70 | 0.35 |
| Color | 0.09 | 0.045 |
| Flavor | 0.43 | 0.215 |
| Total | 100.000 | 50.000 |
| Salivation Region | | |
| Cooked syrup | 96.21 | 48.105 |
| Citric Acid | 1.3 | 0.65 |
| Malic Acid | 1.3 | 0.65 |
| Adipic Acid | 0.42 | 0.21 |
| Succinic Acid | 0.19 | 0.095 |
| “COOLER II,”³ | 0.14 | 0.07 |
| Flavor | 0.44 | 0.22 |
| Total | 100.000 | 50.000 |

¹Partially hydrogenated palm kernel oil
²Mono and diglyceride fatty acid ester sourced from Canola oil (available as “Myverol” 18-99 from Quest, Hoffman Estates, IL.
³A cooling compound available from IFF (Dayton, New Jersey).

### EXAMPLE 3

A confectionery having an isomalt base was prepared as follows using the ingredients set forth in Table 3. For this isomalt confection, the isomalt, sweeteners, acids and water were mixed together and cooked to 170° C. Two batches were prepared simultaneously, and batch #1—contained the acids, while batch #2 contained only the sweeteners, isomalt and water before cooking. After cooking, the hot syrup was cooled to approximately 160° C. and the additional ingredients were added at this time. Flavor and cooling compounds were added to cooked batch #1 and flavor, lipid, surfactant and color were added to cooked batch #2.

The confectionery product was then prepared by dual deposition in the manner described in Example 1.

TABLE 3
Isomalt Candy—Citrus Flavor

| | Ingredient (%) | Ingredient (%) |
|---|---|---|
| | one side | total |
| Cooked Isomalt syrup | | |
| Isomalt - 99.0% | | |
| Water - 1.0% | | |
| Oral Comfort Region | | |
| Cooked Isomalt syrup | 93.15 | 46.58 |
| AceK | 0.04 | 0.02 |
| Sucralose | 0.064 | 0.032 |
| Lipid¹ | 5.59 | 2.8 |
| Surfactant² | 0.70 | 0.35 |
| Colorant | 0.09 | 0.045 |
| Flavorant | 0.37 | 0.0185 |
| Total | 100.000 | 50.000 |
| Salivation Region | | |
| Cooked isomalt syrup | 96.19 | 48.10 |
| AceK | 0.041 | 0.021 |
| Sucralose | 0.066 | 0.033 |
| Citric Acid | 1.30 | 0.65 |
| Malic Acid | 1.30 | 0.65 |
| Adipic Acid | 0.42 | 0.21 |
| Succinic Acid | 0.19 | 0.095 |
| “COOLER”³ | 0.12 | 0.06 |
| Flavorant | 0.38 | 0.19 |
| Total | 100.000 | 50.000 |

¹Partially hydrogenated palm kernel oil
²Mono and diglyceride fatty acid ester sourced from Canola oil (available as “Myverol” 18-99 from Quest, Hoffman Estates, IL.)
³A cooling compound available from IFF (Dayton, New Jersey).

Other variations and modifications of this invention will be obvious to those skilled in this art. This invention is not to be limited except as set forth in the following claims.

What we claim is:

1. A confectionery product comprising:
(i) a salivation region comprising a salivation agent in an amount effective to aid in the stimulation of the flow of saliva in an oral cavity and a salivation region confectionery base; and
(ii) an oral comfort region that is separate and distinct from said salivation region, said oral comfort region comprising an oral comfort ingredient in an amount effective to lubricate, coat or moisten said oral cavity and an oral comfort region confectionery base.

2. The confectionery product according to claim 1, wherein said salivation region confectionery base and said oral comfort region confectionery base are sugar bases.

3. The confectionery product according to claim 2, wherein said sugar bases comprises a sugar selected from the group consisting of sucrose, glucose, fructose, maltose, corn syrup and mixtures thereof.

4. The confectionery product according to claim 1, wherein said salivation region confectionery base and said oral comfort region confectionery base are sugarless bases.

5. The confectionery product according to claim 4, wherein said sugarless bases are selected from group consisting of isomalt, erythritol, hydrogenated starch hydrolysates, sorbitol, xylitol, mannitol and mixture thereof.

6. A confectionery product according to claim 1, wherein said salivation agent is selected from the group consisting of acidulents, cooling compounds, salts, salt enhancers, monosodium glutamate, monosodium glutamate enhancers, flavors and mixtures thereof.

7. A confectionery product according to claim 6, wherein said acidulent is selected from the group consisting of citric acid, malic acid, succinic acid, adipic acid and mixtures thereof.

8. The confectionery product according to claim 1, wherein said salivation agent is present in an amount from about 0.01% to about 4% by weight of the confectionery product.

9. The confectionery product according to claim 1, wherein said oral comfort ingredient is selected from the group consisting of lipids, proteins, surfactants and mixtures thereof.

10. The confectionery product according to claim 9, wherein said oral comfort ingredient is a lipid.

11. A confectionery product according to claim 10, wherein said lipid is present in an amount of about 1 to about 20% by weight of the confectionery product.

12. The confectionery product according to claim 10, wherein said lipid is selected from the group consisting of partially hydrogenated palm kernel oil, medium chain triglycerides, coconut oil, anhydrous milk fat, cocoa butter, corn oil, palm oil, soybean oil, sunflower oil, canola oil and mixtures thereof.

13. The confectionery product according to claim 10, wherein said oral comfort region further comprises a surfactant selected from the group consisting of nonionic surfactants, anionic surfactants and amphoteric surfactants.

14. The confectionery product according to claim 13, wherein said surfactant is present in an amount of about 0.5 percent to about 4 percent by weight of said oral comfort region.

15. The confectionery product according to claim 1, wherein said salivation region comprises an acidulent and a cooling compound.

16. The confectionery product according to claim 15, wherein said cooling compound is present in an amount of about 0.01 to 0.2% by weight of the confectionery product.

17. A method of preparing a confectionery product having a salivation region and oral comfort region that is separate and distinct from said salivation region, said method comprising the steps of:
(i) mixing a salivation region with a salivation region confectionery base to form a salivation agent containing confectionery base;
(ii) mixing an oral comfort ingredient with a oral comfort region confectionery base to form a oral comfort ingredient containing confectionery base; and
(iii) forming said confectionery product with said salivation agent containing confectionery base and said oral comfort ingredient containing confectionery base in a manner that maintains said salivation agent containing confectionery base and said oral comfort ingredient containing confectionery base in separate and distinct regions of said confectionery product.

18. The method according to claim 17, wherein said step of forming comprises substantially simultaneously depositing said salivation agent containing confectionery base and said oral comfort ingredient containing confectionery base in separate and distinct regions of a confectionery mold.

19. The method according to claim 18, wherein said confectionery mold has a raised ridge running along its bottom inner surface that defines said separate and distinct regions of said confectionery mold.

20. A method for treating xerostomia, which comprises:
introducing into the oral cavity a confectionery product containing a confectionery base, a salivation agent concentrated in one region of said product, and an oral comfort ingredient concentrated in a separate region of said product.

21. A method according to claim 20, wherein said salivation agent is an acidulent in combination with a cooling compound.
